(19) United States
(12) Patent Application Publication
(10) Pub. No.: US 2026/0262654 A1
(43) Pub. Date: Sep. 10, 2026

(54) BACTERIAL COMPOSITIONS AND METHODS FOR THE PROTECTION OF TOMATO FROM CLAVIBACTER MICHIGANENSIS SUBSP. MICHIGANENSIS (CMM)

(71) Applicant: THE ROYAL INSTITUTION FOR THE ADVANCEMENT OF LEARNING/MCGILL UNIVERSITY, Montreal, Québec (CA)

(72) Inventor: Mahood FAZLI, Québec (CA)

(21) Appl. No.: 19/168,025

(22) PCT Filed: Mar. 21, 2024
(86) PCT No.: PCT/IB2024/052728

§ 371 Date: Sep. 23, 2025

Related U.S. Application Data
(60) Provisional application No. 63/492,074, filed on Mar. 24, 2023.
(60) Provisional application No. 63/619,441, filed on Jan. 10, 2024.

(51) Int. Cl. A01N 63/22; A01N 59/20; A01P 1/00; A01P 3/00

(52) U.S. Cl. CPC A01N 63/22; A01N 59/20; A01P 1/00; A01P 3/00

(57) ABSTRACT

Described are bacterial compositions and uses thereof for controlling, suppressing and/or preventing an infection from Clavibacter michiganensis subsp. michiganensis (Cmm) in tomato. The bacterial compositions comprise an aqueous mixture of living Bacillus subtilis and living Bacillus pumilus. The bacterial compositions can be combined with existing herbicides, insecticides, fungicides, bactericides, and nutrient, including but not limited copper-containing compounds. Combination with a copper fungicide such as Kocide™ provides a synergistic efficacy.

### CROSS REFERENCE TO RELATED APPLICATION

None

### FIELD OF THE INVENTION

The invention relates to the field of agriculture, and more particularly to prevention and control of bacterial canker (Clavibacter michiganensis pv. michiganensis) in tomato.

### BACKGROUND OF THE INVENTION

Bacterial canker is the most destructive of the bacterial diseases affecting tomato. Bacterial canker is caused by the bacterium Clavibacter michiganensis subsp. michiganensis (Cmm). This microorganism is introduced into plantings primarily via infected seed or transplants and it is capable of spreading rapidly, resulting in devastating losses. This disease is particularly difficult to manage not only because there is no cure, also because the Cmm can be hard to eradicate once it has been introduced into a greenhouse, garden, or field. Therefore, there is an important need for the reduction of economic and environmental costs associated with Cmm infections.

International PCT publication WO 2020/069438 describes using a bacterial culture comprising Bacillus pumilus for protecting tomatoes from Cmm. However, there is still a need for improved methods and compositions for controlling, suppressing and/or preventing infections from Clavibacter michiganensis subsp. michiganensis (Cmm) in tomato.

Particularly, there is a need for methods wherein application of an effective amount bacterial compositions comprising Bacillus subtilis and Bacillus pumilus provide benefits that are measurable in the field. There is also a need for methods in which effective bacterial compositions are contacted with the roots of a tomato plant prior to planting the tomato plant into soil.

There is also a need for greater yield predictability and for a reduction of economic and environmental costs associated with intensive use of currently available canker control agents. Therefore, it would be desirable to provide bacterial compositions and methods for the protection of tomato from Cmm that can be combined with existing herbicides, insecticides, fungicides, bactericides, and nutrient, including but not limited copper-containing compounds.

The present invention addresses these needs and other needs as it will be apparent from the review of the disclosure and description of the features of the invention hereinafter.

### BRIEF SUMMARY OF THE INVENTION

According to one aspect, the invention relates to a bacterial composition comprising an aqueous mixture of living Bacillus subtilis and living Bacillus pumilus, wherein the composition is effective for controlling, suppressing and/or preventing an infection from Clavibacter michiganensis subsp. michiganensis (Cmm) in tomato.

According to another aspect, the invention relates a bacterial composition for use in controlling, suppressing and/or preventing an infection from Clavibacter michiganensis subsp. michiganensis (Cmm) in tomato, wherein said composition comprises an aqueous mixture of living Bacillus subtilis and living Bacillus pumilus.

According to another aspect, the invention relates to a method for controlling, suppressing and/or preventing an infection from Clavibacter michiganensis subsp. michiganensis (Cmm) in tomato, comprising:
providing a bacterial composition comprising at least one of a mixture of Bacillus subtilis and Bacillus pumilus, a culture medium previously inoculated with Bacillus subtilis and Bacillus pumilus, a cell-free extract of Bacillus subtilis and Bacillus pumilus, and/or metabolites produced by Bacillus subtilis and Bacillus pumilus; and
applying an effective amount of said bacterial composition to at least one of a tomato plant, a tomato root, a tomato leaf, a tomato seed, a tomato stem and a tomato fruit.

In embodiments, the method further comprises applying at least one of an herbicide, an insecticide, a fungicide, a bactericide, and a nutrient.

According to another aspect, the invention relates to a method for controlling, suppressing and/or preventing an infection from Clavibacter michiganensis subsp. michiganensis (Cmm) in tomato, comprising:
providing a bacterial composition comprising at least one of a mixture of Bacillus subtilis and Bacillus pumilus, a culture medium previously inoculated with Bacillus subtilis and Bacillus pumilus, a cell-free extract of Bacillus subtilis and Bacillus pumilus, and/or metabolites produced by Bacillus subtilis and Bacillus pumilus;
providing at least one copper fungicide selected from the group consisting of copper sulfate, copper sulfate pentahydrate, copper hydroxide, copper oxychloride sulfate, cuprous oxide, copper octanoate, and mixtures thereof; and
applying an effective amount of said bacterial composition and of said copper fungicide to at least one of a tomato plant, a tomato root, a tomato leaf, a tomato seed, a tomato stem and a tomato fruit.

According to another aspect, the invention relates to a kit for the protection of tomato from Clavibacter michiganensis subsp. michiganensis (Cmm), comprising: (i) a first container comprising a bacterial composition as defined herein; and (ii) a second container comprising at least one of an herbicide, an insecticide, a fungicide, a bactericide, and a nutrient.

According to another aspect, the invention relates to the use of a bacterial composition as defined herein for controlling, suppressing and/or preventing an infection from Clavibacter michiganensis subsp. michiganensis (Cmm) in tomato.

According to another aspect, the invention relates to the combined use of: (i) a bacterial composition comprising a mixture of living Bacillus subtilis and living Bacillus pumilus; and (ii) at least one of an herbicide, an insecticide, a fungicide, a bactericide, and a nutrient, for controlling, suppressing and/or preventing an infection from Clavibacter michiganensis subsp. michiganensis (Cmm) in tomato.

Additional aspects, advantages and features of the present invention will become more apparent upon reading of the following non-restrictive description of preferred embodiments which are exemplary and should not be interpreted as limiting the scope of the invention.

### DETAILED DESCRIPTION OF EMBODIMENTS

The following description provides embodiments by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

The invention aims to address an important need in the prevention and control of bacterial canker in tomato.

As used herein, the terms “Clavibacter michiganensis subsp. Michiganensis” and “Cmm”) are used interchangeably to refer to the Gram-positive, aerobic plant pathogenic bacterium which causes wilt and canker disease in tomato.

As described hereinafter, the present invention proposes new compositions and methods for controlling, suppressing and/or preventing Cmm infections in tomato.

### Bacterial Compositions

One aspect of the invention concerns bacterial compositions that are effective against Cmm. The present invention relies particularly on the combined use of Bacillus subtilis and Bacillus pumilus. Compositions in accordance with the present invention preferably comprises a mixture of living Bacillus subtilis and Bacillus pumilus. In certain embodiments, the invention also encompasses any culture medium previously inoculated with Bacillus subtilis and Bacillus pumilus, cell-free extracts of Bacillus subtilis and Bacillus pumilus, and/or metabolites produced by Bacillus subtilis and Bacillus pumilus.

As used herein, reference to a “culture medium previously inoculated with Bacillus subtilis and Bacillus pumilus” means a medium previously inoculated with the two bacteria, either together or separately. Therefore, this term encompasses mixtures of two or more culture pooled media, previously inoculated with one bacterium and/or both bacteria.

As used herein, reference to a “cell-free extract of Bacillus subtilis and Bacillus pumilus”, refers to a previously inoculated culture medium as defined above from which the bacteria have been removed (e.g., by filtration, by centrifugation, etc.). In embodiments the cell-free extract is a filtered fraction and/or a cell-free supernatant obtained from culturing Bacillus subtilis and/or Bacillus pumilus.

As used herein, reference to “metabolites produced by Bacillus subtilis and Bacillus pumilus” refers to a mixture comprising peptides, proteins, small molecules, lipids etc. which have been produced by the bacteria (synthesis, expression, extraction, secretion, etc.), either separately or in combination.

In embodiments, the Bacillus pumilus consists of “Bacillus pumilus strain deposited with the American Type Culture Collection (ATCC) on Sep. 26, 2018 and bearing deposit number PTA-125304. In embodiments, the Bacillus pumilus consists of strain NES-CAP-1 (GenBank Accession No. MF079281.1).

In embodiments, the Bacillus subtilis consists of strain “Bacillus subtilis strain deposited with the ATCC® Sep. 26, 2018 and bearing deposit number PTA-125303.

In embodiments, the Bacillus subtilis consists of strain Bacillus subtilis strain deposited with the ATCC® on Sep. 26, 2018, and bearing deposit number PTA-125302.

In embodiments, the composition comprises living Bacillus subtilis and living Bacillus pumilus in a 1:1 ratio. In other embodiments, that ratio is 2:1. In other embodiments, that ratio is 1:2.

In embodiments, the composition comprises between 1×10⁹ and 1×10⁶ Bacillus pumilus bacteria/ml. In embodiments, the composition comprises at least 1×10⁹ Bacillus pumilus bacteria/ml, or at least 1×10⁸ Bacillus pumilus bacteria/ml, or at least 1 x 107 Bacillus pumilus bacteria/ml, or at least 1×10⁶ Bacillus pumilus bacteria/ml.

In embodiments, the composition comprises between 1×10⁹ and 1×10⁶ Bacillus subtilis bacteria/ml. In embodiments, the composition comprises at least 1×10⁹ Bacillus subtilis bacteria/ml, or at least 1×10⁸ Bacillus subtilis bacteria/ml, or at least 1×10⁷ Bacillus subtilis bacteria/ml, or at least 1×10⁶ Bacillus subtilis bacteria/ml.

It is also envisionable to include additional bacteria, culture medium, cell-free extracts and/or metabolites thereof, to the composition of the invention. Additional examples of potentially useful microorganisms include, but are not limited to, Lactobacillus spp. (e.g., Lactobacillus helveticus, Lactobacillus rhamnosus, Lactobacillus paracasei, Lactobacillus plantarum, etc.), Lactococcus spp. (e.g., Lactococcus lactis), Bacillus spp. (e.g., Bacillus amyloliquefaciens), Aspergillus spp. (e.g., Aspergillus oryzae), Candida spp. (e.g., Candida utilis), Pseudomonas spp. (e.g., Pseudomonas aeuroginosa, Pseudomonas fluorescens), Saccharomyces spp. (e.g., Saccharomyces cerevisiae), Streptococcus spp. (e.g., Streptococcus lactis), Rhodopseudomonas spp. (e.g., Rhodopseudomonas palustris) and combinations comprising one or more of the above.

The bacterial composition of the invention may further comprise an herbicide, an insecticide, a fungicide, a bactericide, a nutrient, and a mixture thereof.

Examples of potentially useful herbicides include, but are not limited to, Roundup™, atrazine, 2,4-D (Weed-B-Gon™), dicamba (Banvel™), imazethapyr (Pursuit™), metolachlor (Dual™), S-metolachlor (Dual II Magnum™), pendimenthalin (Prowl™), clethodim (Select™), triclopyr (Garlon™), clopyralid (Stinger™), Fluro Star™, flufosinate (Liberty™), halosulfuron (Permit™), isoxaben (Gallery™), sethoxydim (Poast™), bentazon (Basagran™), pyraflufen-ethyl (Epic™), fomesafen (Reflex™), trifluralin (Treflan™), mesotrione (Callisto™), flumioxazin (Valor), dicamba (Banvel™, Clarity™), and imazapyr (Arsenal™).

Examples of potentially useful insecticides include, but are not limited to, pyrethoids, pyrethins, neonicotinoids, organophosphates, carbamates, spinosad, imidacloprid, fipronil, malathion, permethrin, cypermethrin, and chlorpyrifos. Examples of potentially useful bactericide include, but are not limited to, copper sulfate, zinc sulfate, peracetic acid, quaternary ammonium compounds, streptomycin, kasugamycin, tetracycline hydrochloride, phyton 27TM, Aureobasidium pullulans, Actigard™, and Agri-strep™. Examples of potentially useful nutrients include, but are not limited to, nitrogen, phosphorus, potassium, sulphur, magnesium, calcium and other plant micronutrients.

Examples of potentially useful fungicides include, but are not limited to, copper fungicide that includes copper sulfate, copper sulfate pentahydrate, copper hydroxide, copper oxychloride sulfate, cuprous oxide, copper octanoate, and mixtures thereof. Existing commercial examples include, but are not limited to, Kocide 3000™, Kocide 2000™, Cuprofix Ultra 40 Disperss™, Nordox 75 WG™, Champ WG™, Cueva™, Badge SC™, Basic Copper 53™, BOonide™ copper fungicide, Camelot O™, Nu-Cop 3L™, C-O-C-S WDG™, Previsto™, Badge X2™, Badge MAXX™, Cuproxat™ flowable copper fungicide, tribasic copper sulfate, Nordox 30 WG™, Copper-Count-N™ fungicide, Cuprablau Z 35 WP™ and Captain Jack's™ liquid copper fungicide.

In embodiments, the bacterial composition is formulated for application to one or more of a tomato plant, a tomato root, a tomato leaf, a tomato seed, a tomato stem and a tomato fruit.

As described with more details hereinafter, the bacterial compositions in accordance with the present invention may be applied using any suitable method or technique including, but not limited to, seed coating, application to seeds prior to and/or during planting, root inoculation, soil drenching, soil application (e.g., injection), foliar spray (e.g., high- or low-pressure spraying), etc. In embodiments, the applying comprises dipping roots of a tomato plant into the bacterial composition prior to planting the tomato plant into soil.

In one preferred embodiment, the applying comprises soil drenching. As used herein the term “soil drenching” refers to the process of adding compounds or compositions in accordance with the present invention to directly to the base of a plant, preferably to provide deep, targeted treatment.

As used herein, the term “tomato” refers to any one of the more than 10,000 tomato varieties available in the world. The present composition may find useful anti-canker activity against most if not all tomato varieties affected by Cmm, including greenhouse and field-grown varieties. Most common tomato cultivars include but are not limited to, Harris Morgan 1823 (HM 1823), Beefsteak Tomato, Cherokee Purple Tomato, Celebrity Tomato, Brandywine Tomato, San Marzano Tomato, Early Girl Tomato, Black Krim Tomato, Sungold Tomato, Roma Tomato, Better Boy Tomato, Rutgers Tomatom Mortgage Lifter Tomato, Big Beef Tomato, Big Boy Tomato, Black Cherry Tomato, Pineapple Tomato, Amish Paste Tomato, Juliet Tomato, Black Beauty Tomato, Green Zebra Tomato.

Preferably, the bacterial composition and/or method in accordance with the present invention provides at least of the following benefits:
i. increasing tomatoes yield (e.g. higher yield for extra-large tomatoes;
ii. increasing tomatoes size;
iii. increasing gross return of tomatoes per acre;
iv. bioprotection against Cmm;
V. enhancing resistance against Cmm;
vi. reducing damages caused by Cmm;
vii. reducing canker severity and plant tissue affected by canker lesions;
viii. reducing pathological symptoms or lesions resulting from actions of Cmm; and
ix. enhancing plant antimicrobial response against Cmm; and
X. increasing yield for extra-large tomato.

In embodiments, benefit(s) is (are) observed or determined by comparing tomato contacted or not with the bacterial composition of the invention. In embodiments, benefit(s) is (are) observed when comparing tomato contacted with the bacterial composition versus tomatoes contacted with a reference treatment. In embodiments the reference treatment consists of Kocide™ applied as a foliar spray.

Bacterial compositions in accordance with the present invention may also find additional useful preventive and/or curative applications for various agricultural crops (e.g., cereals), fruits (e.g., small fruits, citrus), vegetables, grass, turf, ornamental plants, etc.

The bacterial compositions of the invention may also possibly be useful, for instance, against one or more of the following pathogens: Verticillium dahlia, Sclerotinia sclerotiorum, Fusarium oxysporum f. sp. Lycopersici, Fusarium oxysporum f. sp. Fragariae, Fusarium spp., Rhizoctonia solani, Phytophthora infestans, Pythium spp., Botrytis cinerea, and Alternaria solani.

The bacterial compositions of the invention may also possibly be useful, for the treatment of bacterial, fungal and oomycetes pathogens (blight, mildew, mold, rust, scabs). It could also potentially be useful to control nematodes.

It may also be envisionable to commercialize the bacterial compositions of the invention as a kit for the protection of tomato from Cmm. For instance, a kit in accordance with the present invention may comprise: (i) a first container comprising a bacterial composition as defined herein; and (ii) a second container comprising at least one of an herbicide, an insecticide, a fungicide, a bactericide, and a nutrient. In such kit the content of the first and/or second container may be a ready to use solution or it may be a concentrated solution to be mixed and/or diluted prior use.

### Methods of Protection

Another aspect of the invention concerns methods for protecting tomato from Clavibacter michiganensis subsp. michiganensis (Cmm). In one embodiment, the method comprises applying an effective amount of a bacterial composition as defined herein to a tomato plant, a tomato plant, a tomato root, a tomato leaf, a tomato seed and/or a tomato stem. The present invention encompasses protection of tomato in a variety of plant environments such as fields, greenhouse facilities, vertical farms, urban greening systems, and hydroponic systems.

In embodiments, the method provides for one or more of the benefits listed hereinbefore for the bacterial composition.

The bacterial compositions in accordance with the present invention may be applied using any suitable method including, but not limited to, seed coating, application to seeds prior to and/or during planting, root inoculation, soil drenching, soil application (e.g., injection), foliar spray (e.g., high- or low-pressure spraying), etc.

In embodiments, the applying comprises dipping roots of a tomato plant into the bacterial composition prior to planting the tomato plant into soil.

In one preferred embodiment, the applying comprises soil drenching. As used herein the term “soil drenching” refers to the process of adding compounds or composition(s) in accordance with the present invention directly to the base of a plant, preferably to provide deep, targeted treatment.

In embodiments, the method further comprises applying at least one of an herbicide, an insecticide, a fungicide, a bactericide, and a nutrient.

In particular embodiments, the method further comprises applying at least one copper fungicide. In embodiments, the copper fungicide comprises a compound from copper sulfate, copper sulfate pentahydrate, copper hydroxide, copper oxychloride sulfate, cuprous oxide, copper octanoate, and mixtures thereof. In particular embodiments, the copper fungicide is selected from existing commercial products including, but not limited to, Kocide 3000™, Kocide 2000™, Kocide LF™, Kocide DF™, Kocide 101™, Cuprofix™ Ultra 40 Disperss™, Nordox™, Nordox 75 WG™, Champ WG™, Cueva™, Badge SC™, Basic Copper 53™, Bonide™, Camelot OT, Nu-Cop 3L™, C-O-C-S WDG™, Previsto™, Badge X2™, Badge MAXX™, Cuproxat™, Nordox 30 WG™, Copper-Count-N™ fungicide, Cuprablau Z 35 WP™ and Captain Jack's™ liquid copper fungicide, Bordeaux™ mixture, Cuprofix Disperss™, Champion™, Nu-Cop™, Copper Count-N™, Mastercop Instill™, Phyton™, and CS 2005™

The bacterial composition can be applied at a particular time, or one or more times, depending on parameters including, but not limited to, Cmm population in a tomato plant or soil planted with a tomato plant, environmental conditions, tomato susceptibility, time of planting, etc.

In some embodiments, the bacterial composition is applied: where a plant rooted therein showed a pathological symptom associated with Cmm; where a plant currently rooted therein shows a pathological symptom associated with Cmm; and/or where a tomato plant which will be planted therein is expected to show a pathological symptom associated with Cmm. In some embodiments, the bacterial composition is applied to the seeds that will be planted to such a soil. In some embodiments, the bacterial composition is applied to the seeds from a parent tomato plant that has been planted to such a soil. In some embodiments, bacterial composition is applied to the plant that is rooted in such a soil. In some embodiments, the bacterial composition is applied to a plant that shows a pathological symptom associated with Cmm.

The bacterial composition can be applied after or prior to infection by Cmm. In some embodiments, the composition is applied at least 1 week, 2 weeks, 3 weeks, 1 months, 2 months, 3 months, 4 months, 5 months, or 6 months before planting a seed or plant. In some embodiments, the bacterial composition is applied at least 1 week, 2 weeks, 3 weeks, 1 months, 2 months, or 3 months after planting a seed or plant. In some embodiments, the bacterial composition is applied 1 week, 2 weeks, 3 weeks, 4 weeks, 5 weeks, or 5-10 weeks before harvesting a tomato.

When treating seeds, the bacterial can be applied by a variety of techniques including, but not limited to, high- or low-pressure spraying, coating, immersion, and injection. Once treated, seeds can be planted in natural or artificial soil and cultivated using conventional procedures to produce plants. After plants have propagated from seeds treated with the bacterial composition, the plants may further be treated with one or more applications of the disclosed bacterial composition.

Disclosed bacterial compositions can be applied to all or part of the tomato plant. For example, a disclosed composition can be applied to the stems, roots, leaves, and/or propagules (e.g., cuttings). The plant may be treated at one or more developmental stages. In one embodiment, a disclosed composition is applied to roots.

In some embodiments, the bacterial composition is mixed with or diluted in an agriculturally acceptable carrier before used.

In some embodiments, the bacterial composition is applied to a delivery vehicle, wherein the delivery vehicle serves as a means of transporting the bacteria from the bacterial composition to the soil, plant, seed, field, etc. For example, disclosed bacterial compositions can be applied to a delivery vehicle (e.g., a particle, a polymer, or a substrate) to be used in filtration systems for the treatment of irrigation water. In some embodiments, the bacterial composition is to be applied to a polymer as a wetting agent and/or gel that releases water as needed.

The bacterial composition of the present invention is preferably applied in an amount effective for bioprotection of a tomato from Cmm (e.g., controlling, suppressing and/or preventing a Cmm infection). In some embodiments, the amount is sufficient to prevent Cmm infection. In some embodiments, the amount is sufficient to treat or reduce one or more symptoms associated with Cmm.

In some embodiments, the amount is sufficient to reduce Cmm concentration in a tissue of a tomato plant treated with the bacterial composition. In some embodiments,

Cmm concentration measured in a tissue of a tomato plant 10 days after the step of applying is lower than 10⁹ CFU/g. In some embodiments, Cmm concentration measured in a tissue of a tomato plant 21 days after the step of applying is lower than 10⁹ CFU/g. In some embodiments, Cmm concentration measured in a tissue of a tomato plant 3, 5, 7, 14, 21, 28, 35, or 42 days after the step of applying is lower than 10⁹ CFU/g. In some embodiments, Cmm concentration measured in a tissue of a tomato plant 3, 5, 7, 14, 21, 28, 35, or 42 days after the step of applying is lower than 10⁸ CFU/g. In some embodiments, Cmm concentration measured in a tissue of a tomato plant 3, 5, 7, 14, 21, 28, 35, or 42 days after the step of applying is lower than 10⁷ CFU/g. In some embodiments, Cmm concentration measured in a tissue of a tomato plant 3, 5, 7, 14, 21, 28, 35, or 42 days after the step of applying is lower than 10⁶ CFU/g. In some embodiments,

In some embodiments, Cmm infection is measured in a tissue of a tomato plant by assessing canker severity or canker incidence rated on a 0-100% scale, where 0% displays no disease severity and 100% indicates complete disease takeover of the plant.

Canker control as % of the whole plant may be calculated using Abbott's formula for SAUDPC (i.e., standardized area under the disease progress curve). In some embodiments, the bacterial composition of the present invention is applied in an effective amount for controlling, suppressing and/or preventing canker severity by at least 1%, or by at least 2%, or by at least 3%, or by at least 4%, or by at least 5%, or by at least 6%, or by at least 7%, or by at least 8%, or by at least 9%, or by at least 10% when compared to control untreated tomato plants or crops.

Those skilled in the art will appreciate the fact that specific amounts and required frequency of applications may vary depending on the types and condition of soils, the types and conditions of tomatoes, potency and activity of Cmm, etc. The specific amounts can also vary depending on the environment, for example, whether the plant is in a pot, a greenhouse, a field, etc.

The specific amounts can be determined by using methods known in the art, for example, by testing dose dependent response. In some embodiments, the specific amount is determined by testing dose dependent response on a culture plate with Cmm, for example, by measuring a zone of inhibition. In some embodiments, the specific amount is determined by testing dose dependent response in a pot or in a field. In some embodiments, the specific amount is determined based on the measurement of Cmm concentration or amount of a gene specific Cmm in a tissue of a tomato plant treated with the bacterial composition. In some embodiments, the specific amount is determined based on the concentration of Bacillus pumilus, Bacillus subtilis or both.

In some embodiments, the bacterial culture applied to a tomato plant comprises Bacillus pumilus at a concentration between 106 and 10⁹ CFU/mL, or between 107 and 10⁹ CFU/mL, or between 2.5×10⁷ and 10⁹ CFU/mL, or between 2.5×10⁷ and 8.5×10⁸ CFU/mL, or between 5×10⁷ and 8.5×10⁸ CFU/mL, or between 2×10⁸ and 8.5×10⁸

CFU/mL, or 10⁸ CFU/mL. In some embodiments, the bacterial culture applied to a tomato plant comprises Bacillus subtilis at a concentration between 106 and 10⁹ CFU/mL or 107 and 10⁹ CFU/mL, or between 2.5×10⁷ and 10⁹ CFU/mL, or between 2.5×10⁷ and 8.5×10⁸ CFU/mL, or between 5×10⁷ and 8.5×10⁸ CFU/mL, or between 2×10⁸ and 8.5×10⁸ CFU/mL, or 10⁸ CFU/mL.

In some embodiments, the bacterial composition is applied to the tomato plant to make a final concentration of Bacillus pumilus measured in root, stem or leaf of the tomato plant to range between 106 and 10⁹ CFU/mL, or between 107 and 10⁹ CFU/cm³, or between 2.5×10⁷ and 10⁹ CFU/cm³, or between 2.5×10⁷ and 8.5×10⁸ CFU/cm³, or between 5×10⁷ and 8.5×10⁸ CFU/cm³, or between 2×10⁸ and 8.5×10⁸ CFU/cm³, or between 3×10⁸ and 8×10⁸ CFU/cm³, or 10⁸ CFU/cm³.

In some embodiments, the bacterial composition is applied to the tomato plant to make a final concentration of Bacillus subtilis measured in root, stem or leaf of the tomato plant to range between 106 and 10⁹ CFU/mL, or between 107 and 10⁹ CFU/cm³, or between 2.5×10⁷ and 10⁹ CFU/cm³, or between 2.5×10⁷ and 8.5×10⁸ CFU/cm³, or between 5×10⁷ and 8.5×10⁸ CFU/cm³, or between 2×10⁸ and 8.5×10⁸ CFU/cm³, or between 3×10⁸ and 8×10⁸ CFU/cm³, or 10⁸ CFU/cm³.

In some embodiments, the bacterial composition is applied to the tomato plant to make a final concentration of Bacillus pumilus and Bacillus subtilis measured in root, stem or leaf of the tomato plant to range between 106 and 10⁹ CFU/mL, or between 107 and 10⁹ CFU/cm³, or between 2.5×10⁷ and 10⁹ CFU/cm³, or between 2.5×10⁷ and 8.5×10⁸ CFU/cm³, or between 5×10⁷ and 8.5×10⁸ CFU/cm³, or between 2×10⁸ and 8.5×10⁸ CFU/cm³, or between 3×10⁸ and 8×10⁸ CFU/cm³, or 10⁸ CFU/cm³.

The composition can be applied in an amount that ranges between 0.2 and 3 gal/A, between 0.5 and 2.5 gal/A, between 0.75 and 2 gal/A, 0.5 gal/A, 1 gal/A, 1.25 gal/A, 1.5 gal/A, or 2 gal/A.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following example, which should not be construed as further or specifically limiting.

### Example 1—Evaluation of CXC Effectiveness

Field trials were conducted in Florida over a period of 3 months (September 2022 to December 2022) to evaluate the effectiveness and activity of a composition according to the present invention (referred hereinafter as “CXC™”), against tomato bacterial canker (Clavibacter michiganensis pv. michiganensis) when applied as root dip and soil drench. Activity and effectiveness of CXC were compared to Kocide 3000™, a currently available product against bacterial tomato canker, as well a control untreated check.

### Materials and Methods

### CXC Composition

The bacterial composition tested, referred hereinafter as “CXC” (or as “CXC biocontrol” in Examples 2 and 3), comprised an aqueous mixture of 1) living Bacillus subtilis (ATCC® deposit number PTA-125303) and 2) living Bacillus pumilus (ATCC® deposit number PTA-125304). The two strains of bacteria were present in the mixture at a 1:1 ratio, each strain at a concentration of about 107-109 bacteria/ml. For obtaining the CXC composition, the two microbes were grown separately for at least 72 hours at 28° C. in a shaking incubator (125 rpm) in LB broth comprising tryptone (10 g), yeast extract (5 g), and NaCl (5 g) in 1 liter water. Bacterial number was determined by counting CFU per mL using serial dilution method. Bacterial cultures were serially diluted and aliquots were plated on LB agar medium, incubated at 28 C and CFUs counted after at least 48 hours of incubation. After final harvest, the cultures of the two microbes were mixed together (1:1) and kept in the fridge until use.

### Field Conditions

The trial was conducted on a soil having an overall pH pf 6.6 and an overall Cation Exchange Capacity level of 3.7. Soil composition comprised 0.5% organic matter, 90.4% sand, 4.4% silt and 5.2% clay.

Fresh market tomatoes of the variety Harris Morgan 1823 (HM 1823) were transplanted on Sep. 21, 2022 under simulated commercial conditions (19″ spacing, on 6′ wide beds), to an overall planting density of 4,585 plants/acre. Plots were 32-row-ft on a single bed, replicated four times in a randomized block design and drip-irrigated.

Bacterial canker was inoculated with a backpack sprayer (246 spores/ml) two weeks after planting (Oct. 2, 2022).

CXC treatments were applied once, twice or thrice, as manually poured drenches. The treatments were applied at beginning at planting (designated treatment A, week 0, Sep. 21, 2022) then again 2 weeks later (designated treatment B, week 2, Oct. 6, 2022) and finally again 3 weeks after treatment B (designated treatment C, week 5, Nov. 3, 2022).

A reference treatment of Kocide 3000TM (referred herein as “Kocide”) was applied as foliar sprays at different concentrations (see Table 1 below) using a tractor-drawn spray rig on a weekly basis beginning Oct. 5, 2022 (designated D-K, weeks 2 to 9). The tractor-mounted sprayer operated at 200 PSI.

An untreated check was included for reference (control).

### Evaluations

Canker severity was assessed on ten plants per plot on a 0% to 100% scale, where 100% is a plant with all tissue affected by lesions. Ratings were performed on whole plants, new plant growth, and stems from 10 plants. Evaluations of the whole plant were done on September 30 (9 DA-A), October 7 (2 DA-D), October 15 (3 DA-B), October 21 (2 DA-G), November 9 (7 DA-H), November 22 (7 DA-J), December 1 (8 DA-K), and December 8 (15 DA-K). New plant growth and stem disease evaluation was done on November 3 (1 DA-H). Canker control was calculated using the Abbott's formula from Standardized Area Under Disease Progress Curve (SAUDPC) (average severity over time).

Tomatoes were harvested by hand and organized into five categories: Extra Large, Large, Medium, Sunscald, and Other Culled. Harvests were done on November 23 (8 DA-J) and November 30 (7 DA-K). Counts and weights for marketable small, medium, large, and extra-large tomatoes, and counts alone for unmarketable and Canker-infected tomatoes, were recorded.

### Results

Results for canker severity for the whole plant (%) are shown in Table 1. Canker severity was rated on a 0-100% scale, where 0% displayed no disease severity and 100% indicated complete disease takeover of the crop. Evaluations of the whole plant were done on September 30 (9 DA-A), October 7 (2 DA-D), October 15 (3 DA-B), October 21 (2 DA-G), November 9 (7 DA-H), November 22 (7 DA-J), December 1 (8 DA-K), and December 8 (15 DA-K).

TABLE 1
Canker Severity of the Whole Plant (%)

| Trt | Treatment | | Unit | Appl | 9/30/2022 | 10/7/2022 | 10/15/2022 | 10/21/2022 | 10/26/2022 |
|---|---|---|---|---|---|---|---|---|---|
| No. | Name | Rate | Rate | Code | 9 DA-A | 2 DA-D | 3 DA-B | 2 DA-F | 2 DA-G |
| 1 | CXC | 100 | % v/v | A | 0.0% a | 0.0% a | 0.85% | 2.7% b | 26.3% ab |
| 2 | CXC | 100 | % v/V | AB | 0.0% a | 0.0% a | 0.7% | 2.6% b | 18.9% c |
| 3 | CXC | 100 | % v/v | ABC | 0.0% a | 0.0% a | 0.7% | 3.3% ab | 22.5% bc |
| 4 | Kocide 3000 | 1.5 | lb/a | DEFGHIJK | 0.0% a | 0.0% a | 0.2% | 1,0% c | 27.3% ab |
| S | Untreated Check | | | | 0.0% a | 0.0% a | 1.7% | 4.7% a | 30.3% a |
| | Canker Severity of the Whole Plant (%) | | | | | | | | |
| | Trt | Treatment | | Unit | Appl | 11/9/2022 | 11/22/2022 | 12/1/2022 | 12/8/2022 |
| | No. | Name | Rate | Rate | Code | 7 DA-H | 7 DA-J | 8 DA-K | 15 DA-K |
| | 1 | CXC | 100 | % v/v | A | 23.8% b | 16.9% b | 25.6% c | 49.5% bc |
| | 2 | CXC | 100 | % v/V | AB | 21.5% b | 17.0% b | 23.9% c | 41.5% c |
| | 3 | CXC | 100 | % v/v | ABC | 22.3% b | 13.4% b | 18.5% c | 28.5% d |
| | 4 | Kocide 3000 | 1.5 | lb/a | DEFGHIJK | 30.4% a | 30.6% a | 36.9% b | 54.3% b |
| | S | Untreated Check | | | | 32.9% a | 34.6% a | 51.0% a | 72.9% a |

Results for canker incidence on the whole plant (% of plants) are shown in Table 2. Canker Incidence of the Whole Plant (%). Canker incidence was rated on a 0-100% scale. Evaluations of the whole plant were done on September 30 (9 DA-A), October 7 (2 DA-D), October 15 (3 DA-B), October 21 (2 DA-G), November 9 (7 DA-H), November 22 (7 DA-J), December 1 (8 DA-K), and December 8 (15 DA-K).

TABLE 2
Canker Incidence of the Whole Plant (%)

| Trt | Treatment | | Rate | Appl | Sep. 30, 2022 | Oct. 7, 2022 | Oct. 15, 2022 | Oct. 21, 2022 | Oct. 28, 2022 |
|---|---|---|---|---|---|---|---|---|---|
| No. | Name | Rate | Unit | Code | 9 DA-A | 2 DA-D | 3 DA-B | 2 DA-F | 2 DA-G |
| 1 | CXC | 100 | % v/v | A | 0.0% a | 0.0% a | 27.5% b | 96.0% a | 100.0% a |
| 2 | CXC | 100 | % v/v | AB | 0.0% a | 0.0% a | 22.5% b | 87.5% a | 100.0% a |
| 3 | CXC | 100 | % v/v | ABC | 0.0% a | 0.0% a | 27.5% b | 95.0% a | 100.0% a |
| 4 | Kocide 3000 | 1.5 | lb/a | DEFGHIJK | 0.0% a | 0.0% a | 7.5% c | 47.5% b | 100.0% a |
| 5 | Untreated Check | | | | 0.0% a | 0.0% a | 47.5% a | 92.5% a | 100.0% a |

| Trt | Treatment | | Rate | Appl | Nov. 9, 2022 | Nov. 22, 2022 | Dec. 1, 2022 | Dec. 8, 2022 |
|---|---|---|---|---|---|---|---|---|
| No. | Name | Rate | Unit | Code | 7 DA-H | 7 DA-J | 8 DA-K | 15 DA-K |
| 1 | CXC | 100 | % v/v | A | 100.0% a | 100.0% a | 100.0% a | 100.0% a |
| 2 | CXC | 100 | % v/v | AB | 100.0% a | 100.0% a | 100.0% a | 100.0% a |
| 3 | CXC | 100 | % v/v | ABC | 100.0% a | 100.0% a | 100.0% a | 100.0% a |
| 4 | Kocide 3000 | 1.5 | lb/a | DEFGHIJK | 100.0% a | 100.0% a | 100.0% a | 100.0% a |
| 5 | Untreated Check | | | | 100.0% a | 100.0% a | 100.0% a | 100.0% a |

Canker control as % of the whole plant calculated using Abbott's formula from SAUDPC is shown in Table 3.

TABLE 3
Canker SAUDPC and % Control of the Whole Plant.

| Trt | Treatment | | Rate | Appl | Canker | |
|---|---|---|---|---|---|---|
| No. | Name | Rate | Unit | Code | SAUDPC | % Control |
| 1 | CXC | 100 | % v/v | A | 16.42 c | 36.52% b |
| 2 | CXC | 100 | % v/v | AB | 14.39 cd | 44.52% ab |
| 3 | CXC | 100 | % v/v | ABC | 13.23 d | 48.62% a |
| 4 | Kocide 3000 | 1.5 | lb/a | DEFGHIJK | 21.28 b | 17.34% c |
| 5 | Untreated | | | | 25.86 a | 0.00% d |
| | Check | | | | | |

Results for canker of new plant growth are shown in Table 4. Canker severity was rated on a 0-100% scale, where 0% displayed no disease severity and 100% indicated complete disease takeover of the crop. New plant growth and stem disease evaluation as done on November 3 (1 DA-H).

TABLE 4
Canker of New Plant Growth (%)

| Trt | Treatment | | Rate | Appl | Canker of New Plant Growth | | |
|---|---|---|---|---|---|---|---|
| No. | Name | Rate | Unit | Code | Severity | % Incidence | % Control |
| 1 | CXC | 100 | % v/v | A | 28.90% a | 100% a | 12.61% b |
| 2 | CXC | 100 | % v/v | AB | 19.40% b | 100% a | 41.50% a |
| 3 | CXC | 100 | % v/v | ABC | 21.30% b | 100% a | 35.48% a |
| 4 | Kocide 3000 | 1.5 | lb/a | DEFGHIJK | 33.40% a | 100% a | 4.07% bc |
| 5 | Untreated Check | | | | 33.00% a | 100% a | 0.00% c |

Results for control of canker of stems are shown in Table 5. Canker severity was rated on a 0-100% scale, where 0% displayed no disease severity and 100% indicated complete disease takeover of the crop. New plant growth and stem disease evaluation as done on November 3 (1 DA-H).

TABLE 5
Canker of Stems (%)

| Trt | Treatment | | Rate | Appl | Canker of Stem | | |
|---|---|---|---|---|---|---|---|
| No. | Name | Rate | Unit | Code | Severtiy | Incidence | % Control |
| 1 | CXC | 100 | % v/v | A | 23.00% b | 70.00% b | 27.66% b |
| 2 | CXC | 100 | % v/v | AB | 10.40% c | 40.00% c | 66.70% a |
| 3 | CXC | 100 | % v/v | ABC | 9.10% c | 35.00% c | 71.01% a |
| 4 | Kocide 3000 | 1.5 | lb/a | DEFGHIJK | 20.80% b | 72.50% b | 32.61% b |
| 5 | Untreated Check | | | | 31.90% a | 87.50% a | 0.00% c |

Per-plot count of canker-infected tomatoes is shown in Table 6. Canker 10 infected tomatoes in the field were counted from 50 tomatoes and averaged over the plot on November 20 (5 DA-J), November 25 (2 DA-K), and November 30 (7 DA-K).

TABLE 6
# of Canker Infected Tomatoes (#/plot, averaged from 50 tomatoes)

| Trt | Treatment | | Rate | Appl | Nov. 20, 2022 | Nov. 25, 2022 | Nov. 30, 2022 |
|---|---|---|---|---|---|---|---|
| No. | Name | Rate | Unit | Code | 5 DA-J | 2 DA-K | 7 DA-K |
| 1 | CXC | 100 | % v/v | A | 13.3 ab | 32.0 b | 48.3 b |
| 2 | CXC | 100 | % v/v | AB | 10.3 bc | 24.3 b | 38.5 b |
| 3 | CXC | 100 | % v/v | ABC | 6.8 c | 14.8 c | 18.3 c |
| 4 | Kocide 3000 | 1.5 | lb/a | DEFGHIJK | 11.5 b | 30.8 b | 45.8 b |
| 5 | Untreated Check | | | | 15.5 a | 40.8 a | 63.0 a |

Table 7 and Table 8 show per-plot count (#) and weight (lbs) respectively for Extra-Large tomatoes harvested on November 23 and November 30 Tomatoes were harvested and organized into five categories: Extra Large, Large, Medium, Sunscald, and Other Culled. Harvests were done on November 23 (8 DA-J) and November 30 (7 DA-K).

TABLE 7
Total Harvested Extra Large Tomato Count (#/plot)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 11.3 a | 13.3 a |
| 2 | CXC | 100 | % v/v | AB | 13.8 a | 13.0 a |
| 3 | CXC | 100 | % v/v | ABC | 16.3 a | 19.0 a |
| 4 | Kocide 3000 | 1.5 | lb/a | DEFGHIJK | 12.5 a | 17.8 a |
| 5 | Untreated | | | | 13.0 a | 16.0 a |
| | Check | | | | | |

TABLE 8
Total Harvested Extra Large Tomato Weight (lbs)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 4.39 a | 4.21 a |
| 2 | CXC | 100 | % v/v | AB | 5.49 a | 4.36 a |
| 3 | CXC | 100 | % v/v | ABC | 6.24 a | 6.48 a |
| 4 | Kocide 3000 | 1.5 | lb/a | DEFGHIJK | 4.93 a | 5.06 a |
| 5 | Untreated | | | | 5.14 a | 5.50 a |
| | Check | | | | | |

Table 9 and Table 10 show the per-plot count (#) and weight (lbs) respectively for large tomatoes were harvested and organized into five categories: Extra Large, Large, Medium, Sunscald, and Other Culled. Harvests were done on November 23 (8 DA-J) and November 30 (7 DA-K).

TABLE 9
Total Harvested Large Tomato Count (#/plot)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 31.5 a | 32.3 a |
| 2 | CXC | 100 | % v/v | AB | 30.5 a | 25.3 a |
| 3 | CXC | 100 | % v/v | ABC | 29.0 a | 27.0 a |
| 4 | Kocide 3000 | 1.5 | lb/a | DEFGHIJK | 28.8 a | 29.8 a |
| 5 | Untreated | | | | 30.8 a | 29.0 a |
| | Check | | | | | |

TABLE 10
Total Harvested Large Tomato Count (lbs)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 15.95 a | 13.14 a |
| 2 | CXC | 100 | % v/v | AB | 16.18 a | 10.68 a |
| 3 | CXC | 100 | % v/v | ABC | 14.81 a | 11.79 a |
| 4 | Kocide 3000 | 1.5 | lb/a | DEFGHIJK | 14.59 a | 12.14 a |
| 5 | Untreated | | | | 16.20 a | 12.19 a |
| | Check | | | | | |

5 Table 11 and Table 12 show the per-plot Medium tomato count (#) and total weight (lbs) from the harvests on November 23 and November 30. Harvests were done on November 23 (8 DA-J) and November 30 (7 DA-K).

TABLE 11
Total Harvested Medium Tomato Count (#/plot)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 7.8 a | 14.3 a |
| 2 | CXC | 100 | % v/v | AB | 8.0 a | 13.5 a |
| 3 | CXC | 100 | % v/v | ABC | 8.8 a | 14.8 a |
| 4 | Kocide 3000 | 1.5 | lb/a | DEFGHIJK | 7.8 a | 13.8 a |
| 5 | Untreated | | | | 5.8 a | 15.8 a |
| | Check | | | | | |

TABLE 12
Total Harvested Medium Tomato Count (lbs)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 2.64 a | 4.09 a |
| 2 | CXC | 100 | % v/v | AB | 2.63 a | 4.04 a |
| 3 | CXC | 100 | % v/v | ABC | 2.96 a | 4.15 a |
| 4 | Kocide 3000 | 1.5 | lb/a | DEFGHIJK | 2.50 a | 3.88 a |
| 5 | Untreated | | | | 1.99 a | 4.66 a |
| | Check | | | | | |

Table 13 and Table 14 show per-plot counts of sunscalded and other cull tomatoes respectively, as harvested on November 23 (8 DA-J) and November 30 (7 DA-K).

TABLE 13
Total Harvested Sunscalded Tomato Count (#/plot)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 1.5 a | 2.0 a |
| 2 | CXC | 100 | % v/v | AB | 0.3 a | 0.8 a |
| 3 | CXC | 100 | % v/v | ABC | 0.0 a | 1.3 a |
| 4 | Kocide 3000 | 1.5 | lb/a | DEFGHIJK | 0.3 a | 0.0 a |
| 5 | Untreated | | | | 0.5 a | 1.0 a |
| | Check | | | | | |

TABLE 14
Total Harvested Other Cull Tomato Count (#/plot)

| | | | | | Nov. 23, | Nov. 30, |
|---|---|---|---|---|---|---|
| Trt | Treatment | | Rate | Appl | 2022 | 2022 |
| No. | Name | Rate | Unit | Code | 8 DA-J | 7 DA-K |
| 1 | CXC | 100 | % v/v | A | 5.8 a | 4.8 a |
| 2 | CXC | 100 | % v/v | AB | 6.0 a | 5.0 a |
| 3 | CXC | 100 | % v/v | ABC | 4.3 a | 5.3 a |
| 4 | Kocide 3000 | 1.5 | lb/a | DEFGHIJK | 6.3 a | 6.5 a |
| 5 | Untreated | | | | 4.8 a | 5.5 a |
| | Check | | | | | |

10 Table 15 shows the total per-plot harvested tomato count, by category, from both harvests on November 23 (8 DA-J) and November 30 (7 DA-K).

TABLE 15
Total Harvested Tomato Count (#/plot).

| Trt | Treatment | | Rate | Appl | Total Count | | | | |
|---|---|---|---|---|---|---|---|---|---|
| No. | Name | Rate | Unit | Code | Extra Large | Large | Medium | Sunscald | Other Cull |
| 1 | CXC | 100 | % v/v | A | 24.5 c | 63.8 a | 22.0 a | 3.5 a | 10.5 a |
| 2 | CXC | 100 | % v/v | AB | 26.8 bc | 55.8 a | 21.5 a | 1.0 a | 11.0 a |
| 3 | CXC | 100 | % v/v | ABC | 35.3 a | 56.0 a | 23.5 a | 1.3 a | 9.5 a |
| 4 | Kocide 3000 | 1.5 | lb/a | DEFGHIJK | 30.3 ab | 58.5 a | 21.5 a | 0.3 a | 12.8 a |
| 5 | Untreated Check | | | | 29.0 bc | 59.8 a | 21.5 a | 1.5 a | 10.3 a |

| Trt | Treatment | | Rate | Appl | |
|---|---|---|---|---|---|
| No. | Name | Rate | Unit | Code | Total |
| 1 | CXC | 100 | % v/v | A | 124.3 a |
| 2 | CXC | 100 | % v/v | AB | 116.0 a |
| 3 | CXC | 100 | % v/v | ABC | 125.5 a |
| 4 | Kocide 3000 | 1.5 | lb/a | DEFGHIJK | 123.3 a |
| 5 | Untreated Check | | | | 122.0 a |

Table 16 shows the composition, by category, of the combined harvests done on November 23 and November 30.

TABLE 16
Total Harvested Tomato Composition (%)

| Trt | Treatment | | Rate | Appl | Yield Composition | | | | |
|---|---|---|---|---|---|---|---|---|---|
| No. | Name | Rate | Unit | Code | Extra Large | Large | Medium | Sunscald | Other Cull |
| 1 | CXC | 100 | % v/v | A | 20.38% a | 50.29% a | 18.23% a | 2.51% a | 8.48% a |
| 2 | CXC | 100 | % v/v | AB | 23.29% a | 47.88% a | 18.39% a | 0.97% a | 9.47% a |
| 3 | CXC | 100 | % v/v | ABC | 28.02% a | 44.07% a | 19.14% a | 1.09% a | 7.68% a |
| 4 | Kocide 3000 | 1.5 | lb/a | DEFGHIJK | 24.70% a | 46.89% a | 17.90% a | 0.17% a | 10.35% a |
| 5 | Untreated Check | | | | 24.01% a | 48.06% a | 18.29% a | 1.20% a | 8.44% a |

Table 17 shows the total harvested marketable tomato weight (lbs) by category per plot.

TABLE 17
Total Harvested Marketable Tomato Weight (lbs)

| Trt | Treatment | | Rate | Appl | Total Weight/Plot | | | |
|---|---|---|---|---|---|---|---|---|
| No. | Name | Rate | Unit | Code | Extra Large | Large | Medium | Total |
| 1 | CXC | 100 | % v/v | A | 8.60 b | 29.09 a | 6.73 a | 44.42 a |
| 2 | CXC | 100 | % v/v | AB | 9.85 b | 26.85 a | 6.66 a | 43.36 a |
| 3 | CXC | 100 | % v/v | ABC | 12.71 a | 26.60 a | 7.11 a | 46.43 a |
| 4 | Kocide 3000 | 1.5 | lb/a | DEFGHIJK | 9.99 b | 26.73 a | 6.38 a | 43.09 a |
| 5 | Untreated Check | | | | 10.64 ab | 28.39 a | 6.65 a | 45.68 a |

Table 18 shows estimated marketable return. Estimated harvested marketable tomato weight (MW) was converted to pounds per acre (lbs/acre). Estimated gross income return was calculated by using a value of $18.80 per 25 lbs of tomatoes ($/acre).

TABLE 18
Estimated marketable return

| Trt | Treatment | | Rate | Appl | EST | |
|---|---|---|---|---|---|---|
| No. | Name | Rate | Unit | Code | MW | $/A |
| 1 | CXC | 100 | % v/v | A | 10076.7 a | $7,457 a |
| 2 | CXC | 100 | % v/v | AB | 9837.9 a | $7,280 a |
| 3 | CXC | 100 | % v/v | ABC | 10532.7 a | $7,794 a |
| 4 | Kocide 3000 | 1.5 | lb/a | DEFGHIJK | 9775.5 a | $7,234 a |
| 5 | Untreated Check | | | | 10362.5 a | $7,668 a |

### Discussion

The percentage of plant with any symptoms, reached 100% (all plants) about 4 weeks after infection, with a delay in full infestation on the Kocide-treated plots. Overall, untreated check plots consistently had significantly higher average canker severity than the treated plots, and a positive dose response was observed for the CXC treatments, with the lowest severity average on plots treated three times.

Treatment with the CXC composition, according to a three-application regimen, showed a consistent improvement in control of canker severity and progression, as shown in Tables 1 to 3. Treatment with CXC according to all regimens showed a net improvement in control of canker severity compared, not only to untreated, but also compared to Kocide 3000™, a commercial product currently widely used for this purpose. Accordingly, the composition of the present invention may provide improved canker control and provide greater yield predictability and reduction of economic and environmental costs associated with more intensive use of currently available canker control agents.

Control for canker on new growth and on stems was highest for tomatoes treated with one and two applications of CXC (Tables 4 and 5).

There were significantly more canker infected tomatoes on average at each harvest interval, and the fewest infected tomatoes were collected on the plots treated with the high rate of CXC (Table 6).

As shown in Tables 7 to 14, three applications of CXC produced a larger harvest of extra-large tomatoes (# per plot and total Lbs harvested) than Kocide 3000™ and the untreated check. Further, a single application of CXC produced a larger harvest of large tomatoes (# per plot and total Lbs harvested) than Kocide™ or untreated check. Lastly, 3 applications CXC produced a larger harvest of medium tomatoes (# per plot and total Lbs harvested) than Kocide™

Yields for marketable tomatoes by count were not significantly different and most of the harvest consisted of large or extra-large tomatoes (Tables 15 to 16). However, yield weights were significantly higher for the extra-large tomatoes grown on the plots treated with three applications of CXC (Table 17).

Estimated gross returns were the highest for the treatment comprising three (3) applications of CXC (Table 18).

### Key Findings:

Bacterial canker control was dose dependent for CXC, with significantly greater average control when the treatment was applied three times.

Canker-infected tomato yields were greatest on untreated check or Kocide-treated plots and lowest for CXC applied thrice, thereby demonstrating efficacy of the CXC treatments.

These results also suggest greater yield predictability using the CXC composition.

These results also demonstrate that the CXC composition is a very interesting alternative to currently available canker control agents.

### Example 2—Synergistic Efficacy Kocide™ with the Compositions of the Invention

Field trials were carried out in Thonotosassa, Florida, over a period of 4 months (May 2023 to August 2023) to evaluate to evaluate effectiveness and activity of compositions according to the present invention (referred hereinafter as “CXC ABIO™” and “CXC Biocontrol™”), either solo or in combination with Kocide™, against tomato bacterial canker.

### Materials and Methods

### Tested CXC Compositions

The following bacterial CXC compositions were tested: 1) “CXC Biocontrol™” and 2) “CXC ABIO”. The composition “CXC Biocontrol™” corresponds to the composition “CXC” of Example 1, as defined above. The composition “CXC ABIO” is a biostimulant comprising Bacillin 20.

The Bacillin 20 used for the field trials consisted of a Bacillin 20 aqueous stock solution. Bacillin 20 was purified from Bacillus thuringiensis MS20 strain. The process used to isolate Bacillin 20 involved the following steps: 1) microbial culture production; 2) centrifugation and membrane filtration; 3) butanol extraction of organic metabolites; 4) evaporation of butanol and concentration of extract; 5) fractionation of the organic extract using acetonitrile solvent; 6) purification of Bacillin 20 using HPLC; and 7) mass spectrometry analysis of Bacillin 20 to confirm identity.

During step 1), a starter culture of Bacillus thuringiensis MS20 was prepared and a large-scale production was carried out in a bioreactor. At step 2), bacterial mass was removed by centrifugation and membrane filtration to obtain a cell-free supernatant. The butanol extraction of step 3) was carried out by mixing and vigorous shaking the microbial cell free supernatant with 1-butanol and phase partitioning of 1-butanol. At step 4) butanol was evaporated and an organic extract was obtained by vacuum evaporation of 1-butanol using a rotary evaporator. Next, at step 5), the organic extract was fractionated to remove undesirable and “junk” materials, retaining only active fractions of Bacillin 20 for further purification. At step 6) Bacillin 20 was purified conducting high performance liquid chromatography (HPLC) using a gradient of acetonitrile/water to a target of 85-95% purity of Bacillin 20. At step 7), mass spectrometry analysis was done on a purified sample to identify various fractions using a pure Bacillin 20 as a comparative internal standard.

The purified Bacillin 20 (i.e., about 85 to about 95% purity) was diluted in phosphate buffered saline to obtain a stock solution comprising a 10⁻⁹ M concentration of Bacillin 20. The stock solution of Bacillin 20 was diluted 100-fold in water to a final ready-to-use diluted 10⁻¹¹ M working solution which was used for seed treatment.

Kocide 3000™ (referred herein as “Kocide”) was applied as foliar sprays at a rate of 1.5 lb/a using a tractor-drawn spray rig. The tractor-mounted sprayer operated at 200 PSI. When used in combination with CXC BioControl and/or CCX ABIO, Kocide™ was added to different treatment tanks at the desired rate (see for instance preparation of treatment 4 and treatment 5, as described hereinafter).

### Field Conditions

The trial was conducted on a soil having an overall pH pf 6.4 and an overall Cation Exchange Capacity level of 4. Soil composition comprised 2.1% organic matter, 99.5% sand, substantially no silt and 0.5% clay. Average air temperature ranged between about 22° C. (72° F.) and 30° C. (87° F.), and cumulative measured rainfall was 77.6 cm (30.54 inches).

Tomatoes of the 2255 variety were manually transplanted on May 4 under simulated commercial conditions (19″ spacing, on 6′ wide beds), to an overall planting density of 4,585 plants/acre. CXC Biocontrol was applied as a soil drench May 5, then as soil drenches May 27 and June 23.

CXC ABIO, when used, was applied as a foliar spray application with Kocide™ each week beginning May 5 and continuing through July 18. Treatment 4 was created by diluting 80 ml from the stock solution of CXC ABIO with 8 L of water to create a 10⁻⁹ M concentration and then Kocide™ was added into the tank at a rate of 1.5 LB/A. Treatment 5 was created by withdrawing 80 mL from the tank mixed from treatment 4 at the 10⁻⁹ M concentration, before addition Kocide™. The 80 ml solution drawn from the treatment 4 tank was then mixed with 8 L of water to create a 10⁻¹¹ solution. After the 10⁻¹¹ solution was created, Kocide™ was added to the tank a rate of 1.5 LB/A. An untreated check was included for reference. Plots were 29 row-ft long and replicated four times in a randomized block design. An untreated check was included for reference (control).

### Evaluations

Bacterial canker severity was assessed on whole plants for eleven intervals and on plant tops twice. In mid-June, on whole plants, the incidence of bacterial canker was 100%.

Fifty tomato samples were collected three times and counts of canker-infected fruit were reported.

Fruit was harvested twice and counts and weights for marketable and unmarketable tomatoes were recorded. Yields were uniform.

### Results

Bacterial Canker Severity of the Whole Plant (0-100) is shown in Table 19. Bacterial Canker severity was averaged from ten plants and was rated on a 0-100 scale, where 0 indicates a completely healthy plant and 100 indicates a completely diseased plant. Evaluations were done May 13 (8 DA-A), May 20 (3 DA-E), May 29 (2 DA-B), June 8 (7 DA-G), June 16 (15 AD-G), June 24 (1 DA-C), July 3 (10 DA-C), July 11 (6 DA-J), July 26 (8 DA-K), August 1 (14 DA-K), and August 9 (22 DA-K).

TABLE 19
Bacterial canker severity of the whole plant (0-100)

| Trt | Treatment | | Rate | Appl | May 13, 2023 | May 20, 2023 | May 29, 2023 | Jun. 8, 2023 | Jun. 16, 2023 | Jun. 24, 2023 |
|---|---|---|---|---|---|---|---|---|---|---|
| No. | Name | Rate | Unit | Code | 8 DA-A | 3 DA-E | 2 DA-B | 7 DA-G | 15 DA-G | 1 DA-C |
| 1 | CXC Biocontrol | 100 | % v/v | Dip/Drench | 0.0 a | 2.9 b | 6.0 b | 9.1 b | 27.3 a | 20.6 b |
| 2 | Kocide 3000 | 1.5 | lb/a | Spray | 0.0 a | 0.6 b | 3.9 b | 7.5 b | 31.6 a | 10.9 c |
| 3 | CXC Biocontrol | 100 | % v/v | Dip/Drench | 0.0 a | 0.7 b | 3.5 b | 6.7 b | 30.4 a | 7.8 d |
| | Kocide 3000 | 1.5 | lb/a | Spray | | | | | | |
| 4 | CXC ABIO | 100 | % v/v | Withdrawn | 0.0 a | 1.0 b | 3.5 b | 7.6 b | 31.6 a | 7.0 d |
| | Kocide 3000 | 1.5 | lb/a | Spray | | | | | | |
| 5 | CXC ABIO | 100 | % v/v | Tank Mix | 0.4 a | 3.6 b | 4.0 b | 7.1 b | 32.3 a | 8.1 d |
| | Kocide 3000 | 1.5 | lb/a | Spray | | | | | | |
| 6 | Untreated Check | | | | 0.0 a | 8.7 a | 13.1 a | 15.5 a | 33.9 a | 29.8 a |

| Trt | Treatment | | Rate | Appl | Jul. 3, 2023 | Jul. 11, 2023 | Jul. 26, 2023 | Aug. 1, 2023 | Aug. 9, 2023 |
|---|---|---|---|---|---|---|---|---|---|
| No. | Name | Rate | Unit | Code | 10 DA-C | 6 DA-J | 8 DA-K | 14 DA-K | 22 DA-K |
| 1 | CXC Biocontrol | 100 | % v/v | Dip/Drench | 21.6 b | 24.9 b | 16.3 b | 17.9 b | 20.6 bc |
| 2 | Kocide 3000 | 1.5 | lb/a | Spray | 15.8 c | 15.9 c | 8.6 c | 11.9 c | 15.3 c |
| 3 | CXC Biocontrol | 100 | % v/v | Dip/Drench | 13.3 c | 14.0 c | 5.6 d | 8.1 c | 13.5 c |
| | Kocide 3000 | 1.5 | lb/a | Spray | | | | | |
| 4 | CXC ABIO | 100 | % v/v | Withdrawn | 12.4 c | 9.3 d | 15.8 b | 20.0 b | 23.6 b |
| | Kocide 3000 | 1.5 | lb/a | Spray | | | | | |
| 5 | CXC ABIO | 100 | % v/v | Tank Mix | 12.3 c | 9.4 d | 16.8 b | 20.3 b | 23.4 b |
| | Kocide 3000 | 1.5 | lb/a | Spray | | | | | |
| 6 | Untreated Check | | | | 30.9 a | 35.0 a | 26.4 a | 42.5 a | 47.0 a |

Table 20 shows bacterial canker incidence of the whole plant as a percentage. Bacterial canker severity was averaged from ten plants and was rated on a 0-100 scale, where 0 indicates a completely healthy plant and 100 indicates a completely diseased plant. Evaluations were done May 13 (8 DA-A), May 20 (3 DA-E), May 29 (2 DA-B), June 8 (7 DA-G), June 16 (15 AD-G), June 24 (1 DA-C), July 3 (10 DA-C), July 11 (6 DA-J), July 26 (8 DA-K), August 1 (14 DA-K), and August 9 (22 DA-K).

TABLE 20
Bacterial canker incidence of the whole plant (%)

| | | | | | | May | May | | | |
|---|---|---|---|---|---|---|---|---|---|---|
| Trt | Treatment | | Rate | Appl | May 13, 2023 | 20, 2023 | 29, 2023 | Jun. 8, 2023 | Jun. 16, 2023 | Jun. 24, 2023 |
| No. | Name | Rate | Unit | Code | 8 DA-A | 3 DA-E | 2 DA-B | 7 DA-G | 15 DA-G | 1 DA-C |
| 1 | CXC Biocontrol | 100 | % v/v | Dip/Drench | 0.0% a | 50.0% ab | 77.5% a | 92.5% a | 100.0% a | 100.0% a |
| 2 | Kocide 3000 | 1.5 | lb/a | Spray | 0.0% a | 17.5% b | 57.5% a | 100.0% a | 100.0% a | 100.0% a |
| 3 | CXC Biocontrol | 100 | % v/v | Dip/Drench | 0.0% a | 15.0% b | 37.5% a | 95.0% a | 100.0% a | 92.5% ab |
| | Kocide 3000 | 1.5 | lb/a | Spray | | | | | | |
| 4 | CXC ABIO | 100 | % v/v | Withdrawn | 0.0% a | 25.0% b | 57.5% a | 97.5% a | 100.0% a | 90.0% a |
| | Kocide 3000 | 1.5 | lb/a | Spray | | | | | | |
| 5 | CXC ABIO | 100 | % v/v | Tank Mix | 5.0% a | 40.0% b | 62.5% a | 97.5% a | 100.0% a | 100.0% a |
| | Kocide 3000 | 1.5 | lb/a | Spray | | | | | | |
| 6 | Untreated Check | | | | 0.0% a | 87.5% a | 95.0% a | 100.0% a | 100.0% a | 100.0% a |

| Trt | Treatment | | Rate | Appl | Jul. 3, 2023 | Jul. 11, 2023 | Jul. 26, 2023 | Aug. 1, 2023 | Aug. 9, 2023 |
|---|---|---|---|---|---|---|---|---|---|
| No. | Name | Rate | Unit | Code | 10 DA-C | 6 DA-J | 8 DA-K | 14 DA-K | 22 DA-K |
| 1 | CXC Biocontrol | 100 | % v/v | Dip/Drench | 100.0% a | 100.0% a | 100.0% a | 100.0% a | 100.0% a |
| 2 | Kocide 3000 | 1.5 | lb/a | Spray | 100.0% a | 100.0% a | 100.0% a | 100.0% a | 100.0% a |
| 3 | CXC Biocontrol | 100 | % v/v | Dip/Drench | 100.0% a | 100.0% a | 90.0% a | 100.0% a | 100.0% a |
| | Kocide 3000 | 1.5 | lb/a | Spray | | | | | |
| 4 | CXC ABIO | 100 | % v/v | Withdrawn | 100.0% a | 97.5% a | 97.5% a | 100.0% a | 100.0% a |
| | Kocide 3000 | 1.5 | lb/a | Spray | | | | | |
| 5 | CXC ABIO | 100 | % v/v | Tank Mix | 100.0% a | 100.0% a | 100.0% a | 100.0% a | 100.0% a |
| | Kocido 3000 | 1.5 | lb/a | Spray | | | | | |
| 6 | Untreated Check | | | | 100.0% a | 100.0% a | 100.0% a | 100.0% a | 100.0% a |

Table 21 shows bacterial canker of the whole plant SAUDPC and as a percentage of the control group.

TABLE 21
Bacterial canker of the whole plant (SAUDPC and % of control)

| Trt | Treatment | | Rate | Appl | BC of Whole Plant | |
|---|---|---|---|---|---|---|
| No. | Name | Rate | Unit | Code | SAUDPC | % Control |
| 1 | CXC | 100 | % v/v | Dip/ | 15.94 b | 38.36% c |
| | Biocontrol | | | Drench | | |
| 2 | Kocide 3000 | 1.5 | lb/a | Spray | 11.43 cd | 56.00% ab |
| 3 | CXC | 100 | % v/v | Dip/ | 9.68 d | 62.80% a |
| | Biocontrol | | | Drench | | |
| | Kocide 3000 | 1.5 | lb/a | Spray | | |
| 4 | CXC ABIO | 100 | % v/v | Withdrawn | 11.75 c | 54.72% b |
| | Kocide 3000 | 1.5 | lb/a | Spray | | |
| 5 | CXC ABIO | 100 | % v/v | Tank Mix | 12.29 c | 52.55% b |
| | Kocide 3000 | 1.5 | lb/a | Spray | | |
| 6 | Untreated | | | | 25.97 a | 0.00% d |
| | Check | | | | | |

Table 22 shows bacterial canker severity of the plant top. Bacterial canker severity was averaged from ten plants and was rated on a 0-100 scale, where 0 indicates a completely healthy plant and 100 indicates a completely diseased plant. Evaluations were June 16 (15 DA-G) and July 19 (75 DA-A).

TABLE 22
Bacterial canker severity of the plant top (0-100)

| | | | | | Jun. 16, | Jul. 19, |
|---|---|---|---|---|---|---|
| Trt | Treatment | | Rate | Appl | 2023 | 2023 |
| No. | Name | Rate | Unit | Code | 15 DA-G | 75 DA-A |
| 1 | CXC Biocontrol | 100 | % v/v | Dip/Drench | 11.4 b | 3.5 c |
| 2 | Kocide 3000 | 1.5 | lb/a | Spray | 7.8 bc | 3.8 c |
| 3 | CXC Biocontrol | 100 | % v/v | Dip/Drench | 4.5 c | 4.3 bc |
| | Kocide 3000 | 1.5 | lb/a | Spray | | |
| 4 | CXC ABIO | 100 | % v/v | Withdrawn | 7.1 bc | 4.1 c |
| | Kocide 3000 | 1.5 | lb/a | Spray | | |
| 5 | CXC ABIO | 100 | % v/v | Tank Mix | 5.4 c | 6.9 b |
| | Kocide 3000 | 1.5 | lb/a | Spray | | |
| 6 | Untreated Check | | | | 27.0 a | 13.3 a |

Table 23 shows bacterial canker percent incidence of the plant top. Bacterial canker incidence was averaged from ten plants and was rated on a 0-100 scale, where 0 indicates a completely healthy plant and 100 indicates a completely diseased plant. Evaluations were June 16 (15 DA-G) and July 19 (75 DA-A).

TABLE 23
Bacterial canker incidence of the plant top (%)

| | | | | | Jun. 16, | Jul. 19, |
|---|---|---|---|---|---|---|
| Trt | Treatment | | Rate | Appl | 2023 | 2023 |
| No. | Name | Rate | Unit | Code | 15 DA-G | 75 DA-A |
| 1 | CXC Biocontrol | 100 | % v/v | Dip/Drench | 95.0% a | 57.5% c |
| 2 | Kocide 3000 | 1.5 | lb/a | Spray | 87.5% a | 62.5% bc |
| 3 | CXC Biocontrol | 100 | % v/v | Dip/Drench | 80.0% a | 60.0% c |
| | Kocide 3000 | 1.5 | lb/a | Spray | | |
| 4 | CXC ABIO | 100 | % v/v | Withdrawn | 90.0% a | 67.5% bc |
| | Kocide 3000 | 1.5 | lb/a | Spray | | |
| 5 | CXC ABIO | 100 | % v/v | Tank Mix | 90.0% a | 85.0% ab |
| | Kocide 3000 | 1.5 | lb/a | Spray | | |
| 6 | Untreated Check | | | | 100.0% a | 100.0% a |

Table 24 shows bacterial canker incidence of the of the plant top SAUDPC and as a percentage of the control group.

TABLE 24
Bacterial canker incidence of the plant top (SAUDPC and % of control)

| Trt | Treatment | | Rate | Appl | BC of Plant Top | |
|---|---|---|---|---|---|---|
| No. | Name | Rate | Unit | Code | SAUDPC | % Control |
| 1 | CXC | 100 | % v/v | Dip/ | 7.44 b | 62.74% b |
| | Biocontrol | | | Drench | | |
| 2 | Kocide 3000 | 1.5 | lb/a | Spray | 5.75 bc | 71.62% ab |
| 3 | CXC | 100 | % v/v | Dip/ | 4.38 c | 78.13% a |
| | Biocontrol | | | Drench | | |
| | Kocide 3000 | 1.5 | lb/a | Spray | | |
| 4 | CXC ABIO | 100 | % v/v | Withdrawn | 5.63 bc | 72.32% ab |
| | Kocide 3000 | 1.5 | lb/a | Spray | | |
| 5 | CXC ABIO | 100 | % v/v | Tank Mix | 6.14 bc | 69.36% ab |
| | Kocide 3000 | 1.5 | lb/a | Spray | | |
| 6 | Untreated | | | | 20.13 a | 0.00% c |
| | Check | | | | | |

Table 25 shows canker infected fruit count (number of fruit per plot). Fruit infected with bacterial canker prior to harvest was counted. Counts were taken July 17 (12 DA-J), July 25 (7 DA-K), and August 3 (16 DA-K).

TABLE 25
Canker infected fruit count (#/plot)

| Trt | Treatment | | Rate | Appl | Jul. 17, 2023 | Jul. 25, 2023 | Aug. 3, 2023 | |
|---|---|---|---|---|---|---|---|---|
| No. | Name | Rate | Unit | Code | 12 DA-J | 7 DA-K | 16 DA-K | Total |
| 1 | CXC Biocontrol | 100 | % v/v | Dip/Drench | 3.8 ab | 8.0 c | 12.0 c | 23.8 c |
| 2 | Kocide 3000 | 1.5 | lb/a | Spray | 1.0 c | 5.5 d | 9.3 d | 15.8 d |
| 3 | CXC Biocontrol | 100 | % v/v | Dip/Drench | 3.5 b | 3.0 e | 6.5 e | 13.0 e |
| | Kocide 3000 | 1.5 | lb/a | Spray | | | | |
| 4 | CXC ABIO | 100 | % v/v | Withdrawn | 4.0 ab | 12.5 b | 16.8 b | 33.3 b |
| | Kocide 3000 | 1.5 | lb/a | Spray | | | | |
| 5 | CXC ABIO | 100 | % v/v | Tank Mix | 3.5 b | 13.3 b | 17.8 b | 34.5 b |
| | Kocide 3000 | 1.5 | lb/a | Spray | | | | |
| 6 | Untreated Check | | | | 5.0 a | 18.0 a | 24.0 a | 47.0 a |

Table 26 shows the percentage of Canker infected fruit. Fruit infected with bacterial canker prior to harvest was counted. Counts were taken July 17 (12 DA-J), July 25 (7 DA-K), and August 3 (16 DA-K).

TABLE 26
Canker infected fruit (%)

| Trt | Treatment | | Rate | Appl | Jul. 17, 2023 | Jul. 25, 2023 | Aug. 3, 2023 | |
|---|---|---|---|---|---|---|---|---|
| No. | Name | Rate | Unit | Code | 12 DA-J | 7 DA-K | 16 DA-K | Total |
| 1 | CXC Biocontrol | 100 | % v/v | Dip/Drench | 7.5% ab | 16.0% c | 24.0% c | 15.8% c |
| 2 | Kocide 3000 | 1.5 | lb/a | Spray | 2.0% c | 11.0% d | 18.5% d | 10.5% d |
| 3 | CXC Biocontrol | 100 | % v/v | Dip/Drench | 7.0% b | 6.0% e | 13.0% e | 8.7% e |
| | Kocide 3000 | 1.5 | lb/a | Spray | | | | |
| 4 | CXC ABIO | 100 | % v/v | Withdrawn | 8.0% ab | 25.0% b | 33.5% b | 22.2% b |
| | Kocide 3000 | 1.5 | lb/a | Spray | | | | |
| 5 | CXC ABIO | 100 | % v/v | Tank Mix | 7.0% b | 26.5% b | 35.5% b | 23.0% b |
| | Kocide 3000 | 1.5 | lb/a | Spray | | | | |
| 6 | Untreated Check | | | | 10.0% a | 36.0% a | 48.0% a | 31.3% a |

### Discussion

Untreated check plants had significantly higher average pest severity (Table 19). The untreated plots had significantly more canker infected fruit, and the plants treated with both CXC Biocontrol™ and Kocide™ had the fewest (Tables 25 and 26).

Combining Kocide™ and CXC Biocontrol™ resulted in significantly better canker suppression relative to either treatment alone (Tables 25 and 26).

These results show that combined treatment with CXC Biocontrol™ and Kocide achieves better results than treatment with either CXC Biocontrol™ or Kocide Alone™, indicating a synergistic effect of the treatments.

On plant tops, canker disease incidence was lowest with the CXC biocontrol (Treatment 1). Withdrawn treatment (Treatment 4) was the second most effective at reducing canker incidence followed by Treatment 5 (tank mix alone) (Table 23).

Overall, these results clearly demonstrate that compositions in accordance with the present invention are efficacious and can successfully improve and/or complement current existing means of canker control. Therefore, the compositions and methods in accordance with the present invention may also be useful for reducing economic and environmental costs associated with intensive use of existing commercially popular canker control agents.

### Example 3—Beneficial Effects of CXC Biocontrol™ Applied as a Drench and Foliar Spray, when Used Alone or in Combination with Kocide™

Field trials were carried out in Thonotosassa, Florida, over a period of 4 months (October 2023 to February 2024) to evaluate to evaluate effectiveness and activity of compositions according to the present invention (referred hereinafter as “CXC Biocontrol™”), when applied as a drench and/or as a foliar spray, either solo or in combination with Kocide™, against tomato bacterial canker.

### Materials and Methods

### Treatments

The following treatments were tested for this study:
1. Untreated Control
2. CXC Biocontrol 1% v/v (A, Root Dip), CXC Biocontrol 0.01% (B-N, AA-AC, Drench)
3. Kocide™ 1.5 lb/a (O-Z, AD-AG, Foliar)
4. CXC Biocontrol 1% v/v (A, Root Dip), CXC Biocontrol 0.01% (CEGIKM, Drench), Kocide™ 1.5 lb/a (PRTVXZ)
5. CXC Biocontrol 1% v/v (O-Z AD-AG, Foliar), Kocide™ 1.5 lb/a (O-Z, AD-AG, Foliar)
6. CXC Biocontrol 1% v/v (A, Root Dip), CXC Biocontrol 0.1% v/v (DHL, Drench), Actigard™ 0.75 oz/a (QUY, Foliar), Firewall™ 200 ppm pr (RVZ, Foliar), Kocide™ 1.5 lb/a (PTX, Foliar)

The “CXC Biocontrol™” correspond to the composition “CXC” of Example 1, as defined above.

### Experimental Unit

1823 variety tomatoes were manually transplanted into 6′ wide rows spaced 19″ apart on Oct. 17, 2023. Roots were dipped in 1% v/v CXC Biocontrol™ prior to transplant as the first application. Applications B to N and AA to AC were applied as a soil drench directly onto the base of the plant. Remaining applications (O to Z, AD to AG) were applied via a tractor mounted Nifty Fifty sprayer with an operating pressure of 200 PSI. Additional applications of AA-AG were added to the original spray program to extend applications to harvest. Treatments included CXC Biocontrol, Kocide, CXC Biocontrol applied as a foliar with Kocide or as a drench with Kocide, and CXC Biocontrol in rotation with Actigard, Firewall and Kocide. An untreated check was also included for reference. Plots were 25′ long and replicated four times in a randomized block design.

### Application Equipment

Application A was a root dip prior to transplant. Application B-N and AA-AC were applied as soil drench directly to the base of plant. Application O-Z and AD-AG were foliar sprays applied utilizing a tractor mounted nifty-fifty operating at 200 psi.

### Evaluations

Bacterial Canker severity was averaged from ten plants and was rated on a 0-100 scale, where 0 indicates a completely healthy plant and 100 indicates a completely diseased plant. Evaluations were done on October 28 (5 DA-P), November 3 (4 DA-Q), November 9 (3 DA-R), November 18 (5 DA-S), November 23 (3 DA-T), November 30 (3 DA-U), December 10 (6 DA-V), December 21 (3 DA-X), December 28 (2 DA-Y), January 5 (3 DA-Z), January 15 (7 DA-AD), and January 22 (7 DA-AE).

Bacterial speck severity was evaluated on December 29 (3 DA-Y) on a 0-100 scale, averaged from ten plants per plot).

Tomatoes were harvested three times over the season for extra-large, large, medium, worm infested, sunscalded, and bacterial canker infected tomatoes. Harvests were done on January 19 (4 DA-AA), February 1 (3 DA-AG), and February 12 (14 DA-AG).

### Results

Bacterial Canker Severity of the Whole Plant (0-100) is shown in Table 27. Bacterial Canker severity was averaged from ten plants and was rated on a 0-100 scale, where 0 indicates a completely healthy plant and 100 indicates a completely diseased plant. Evaluations were done on October 28 (5 DAP), November 3 (4 DA-Q), November 9 (3 DA-R), November 18 (5 DA-S), November 23 (3 DA-T), November 30 (3 DA-U), December 10 (6 DA-V), December 21 (3 DA-X), December 28 (2 DA-Y), January 5 (3 DA-Z), January 15 (7 DA-AD), and January 22 (7 DA-AE).

TABLE 27
Bacterial canker severity of the whole plant (0-100)

| | | | | | | Oct. 28, | Nov. 3, | Nov. 9, | Nov. 15, | Nov. 23, | Nov. 30, |
|---|---|---|---|---|---|---|---|---|---|---|---|
| Trt | Treatment | | Rate | Appl | Trt | 2023 | 2023 | 2023 | 2023 | 2023 | 2023 |
| No. | Name | Rate | Unit | Code | Descr. | 5 DA-P | 4 DA-Q | 3 DA-R | 5 DA-S | 3 DA-T | 3 DA-U |
| 1 | Untreated Control | | | | | 2.4 a | 2.8 a | 3.8 a | 8.4 a | 10.5 a | 14.1 a |
| 2 | CXC Biocontrol | 1 | % v/v | A | Root Dip | 0.3 c | 0.4 b | 0.5 b | 2. [symbol] a | 2.8 b | 6.4 bc |
| | CXC Biocontrol | 0.01 | % v/v | B-N | Drench | | | | | | |
| 3 | Kocide | 1.5 | lb/a | O-Z | Foliar | 0.4 bc | 0.8 b | 0.6 b | 1.5 a | 1.9 b | 4.9 c |
| 4 | CXC Biocontrol | 1 | % v/v | A | Root Dip | 0.4 bc | 0.3 b | 0.9 b | 4.5 a | 4.7 b | 8.7 b |
| | CXC Biocontrol | 0.01 | % v/v | CEGIKM | Drench | | | | | | |
| | Kocide | 1.5 | lb/a | PRTVXZ | Foliar | | | | | | |
| 5 | CXC Biocontrol | 1 | % v/v | O-Z | Foliar | 0.9 b | 1. [symbol] ab | 1.9 b | 5.0 a | 5.0 b | 5.8 c |
| | Kocide | 1.5 | lb/a | O-Z | Foliar | | | | | | |
| 6 | CXC Biocontrol | 1 | % v/v | A | Root Dip | 0.7 bc | 1.3 ab | 2.1 ab | 5.6 a | 4.9 b | 6.9 bc |
| | CXC Biocontrol | 0.01 | % v/v | DHL | Drench | | | | | | |
| | Actigard | 0.75 | oz/a | QUY | Foliar | | | | | | |
| | Firewall | 200 | ppm pr | RVZ | Foliar | | | | | | |
| | Kocide | 1.5 | lb/a | PTX | Foliar | | | | | | |
| | | | | | | Dec. 10, | Dec. 21, | Dec. 28, | Jan. 5, | Jan. 15, | Jan. 22, |
| Trt | Treatment | | Rate | Appl | Trt | 2023 | 2023 | 2023 | 2024 | 2024 | 2024 |
| No. | Name | Rate | Unit | Code | Descr. | 6 DA-V | 3 DA-X | 2 DA-Y | 3 DA-Z | 7 DA-AD | 7 DA-AE |
| 1 | Untreated Control | | | | | 13.6 a | 14.4 a | 17.9 a | 21.3 a | 20.5 a | 29.5 a |
| 2 | CXC Biocontrol | 1 | % v/v | A | Root Dip | 2.8 c | 3.4 c | 3.8 d | 6.0 d | 6.5 c | 8.6 d |
| | CXC Biocontrol | 0.01 | % v/v | B-N | Drench | | | | | | |
| 3 | Kocide | 1.5 | lb/a | O-Z | Foliar | 8.8 b | 9.0 b | 12.5 b | 16.3 b | 10.6 b | 15.4 b |
| 4 | CXC Biocontrol | 1 | % v/v | A | Root Dip | 4.5 c | 4.3 c | 5.3 cd | 7.9 cd | 6.6 c | 8.3 b |
| | CXC Biocontrol | 0.01 | % v/v | CEGIKM | Drench | | | | | | |
| | Kocide | 1.5 | lb/a | PRTVXZ | Foliar | | | | | | |
| 5 | CXC Biocontrol | 1 | % v/v | O-Z | Foliar | 9.5 b | 8.5 b | 11.5 b | 15.1 b | 10.9 b | 14.1 bc |
| | Kocide | 1.5 | lb/a | O-Z | Foliar | | | | | | |
| 6 | CXC Biocontrol | 1 | % v/v | A | Root Dip | 5.1 c | 6.5 bc | 8.6 bc | 11.0 c | 9.1 bc | 10.8 cd |
| | CXC Biocontrol | 0.01 | % v/v | DHL | Drench | | | | | | |
| | Actigard | 0.75 | oz/a | QUY | Foliar | | | | | | |
| | Firewall | 200 | ppm pr | RVZ | Foliar | | | | | | |
| | Kocide | 1.5 | lb/a | PTX | Foliar | | | | | | |

[symbol]

indicates data missing or illegible when filed

As shown in Table 27, pest pressure was relatively low for the duration of the trial, the highest observed in the untreated check.

Plots treated with just CXC Biocontrol had significantly better control over bacterial canker followed by plots treated with CXC Biocontrol applied as a drench with Kocide (Table 28).

TABLE 28
Bacterial Canker SAUDPC and % Control of the Whole Plant.

| Trt | Treatment | | Rate | Appl | Trt | Bacterial Canker | |
|---|---|---|---|---|---|---|---|
| No. | Name | Rate | Unit | Code | Descr. | SAUDPC | % Control |
| 1 | Untreated Control | | | | | 13.59 a | 0.00% e |
| 2 | CXC Biocontol | 1 | % v/v | A | Root Dip | 3.75 d | 72.15% a |
| | CXC Biocontrol | 0.01 | % v/v | B-N | Drench | | |
| 3 | Kocide | 1.5 | lb/a | O-Z | Foliar | 7.31 b | 45.65% cd |
| 4 | CXC Biocontrol | 1 | % v/v | A | Root Dip | 4.93 cd | 63.53% ab |
| | CXC Biocontrol | 0.01 | % v/v | CEGIKM | Drench | | |
| | Kocide | 1.5 | lb/a | PRTVXZ | Foliar | | |
| 5 | CXC Biocontrol | 1 | % v/v | O-Z | Foliar | 7.93 b | 41.19% d |
| | Kocide | 1.5 | lb/a | O-Z | Foliar | | |
| 6 | CXC Biocontrol | 1 | % v/v | A | Root Dip | 6.32 bc | 53.33% bc |
| | CXC Biocontrol | 0.01 | % v/v | DHL | Drench | | |
| | Actigard | 0.75 | oz/a | QUY | Foliar | | |
| | Firewall | 200 | ppm pr | RVZ | Foliar | | |
| | Kocide | 1.5 | lb/a | PTX | Foliar | | |

Bacterial speck was observed on December 29 (3 DA-Y). CXC Biocontrol solo demonstrated to provide the best control against bacteria for the one day observation (Table 29).

TABLE 29
Bacterial Speck % Control of the Whole Plant (%)

| | | | | | | Dec. 29, |
|---|---|---|---|---|---|---|
| Trt | Treatment | | Rate | Appl | Trt | 2023 |
| No. | Name | Rate | Unit | Code | Descr. | 3 DA-Y |
| 1 | Untreated | | | | | 0.00% d |
| | Control | | | | | |
| 2 | CXC Biocontol | 1 | % v/v | A | Root Dip | 74.02% a |
| | CXC Biocontrol | 0.01 | % v/v | B-N | Drench | |
| 3 | Kocide | 1.5 | lb/a | O-Z | Foliar | 16.80% c |
| 4 | CXC Biocontrol | 1 | % v/v | A | Root Dip | 33.98% b |
| | CXC Biocontrol | 0.01 | % v/v | CEGIKM | Drench | |
| | Kocide | 1.5 | lb/a | PRTVXZ | Foliar | |
| 5 | CXC Biocontrol | 1 | % v/v | O-Z | Foliar | 5.22% cd |
| | Kocide | 1.5 | lb/a | O-Z | Foliar | |
| 6 | CXC Biocontrol | 1 | % v/v | A | Root Dip | 40.44% b |
| | CXC Biocontrol | 0.01 | % v/v | DHL | Drench | |
| | Actigard | 0.75 | oz/a | QUY | Foliar | |
| | Firewall | 200 | ppm pr | RVZ | Foliar | |
| | Kocide | 1.5 | lb/a | PTX | Foliar | |

Plots were harvested three times for extra-large, large, medium, sunscalded, worm infested, and bacterial canker infected fruit. Kocide and CXC Biocontrol in rotation had the fewest bacterial canker infected fruit (Table 30).

TABLE 30
Total Harvested Tomato Count (#/plot)

| Trt | Treatment | | Rate | Appl | Trt | Total Count | | | | | |
|---|---|---|---|---|---|---|---|---|---|---|---|
| No. | Name | Rate | Unit | Code | Descr. | Extra Large | Large | Medium | Worm | Sunscalded | Bacterial |
| 1 | Untreated Control | | | | | 100.3 a | 213.8 a | 147.5 a | 10.8 a | 8.0 a | 44.8 a |
| 2 | CXC Biocontol | 1 | % v/v | A | Root Dip | 119.0 a | 228.5 a | 161.3 a | 17.5 a | 8.0 a | 20.5 b |
| | CXC Biocontrol | 0.01 | % v/v | B-N | Drench | | | | | | |
| 3 | Kocide | 1.5 | lb/a | O-Z | Foliar | 96.0 a | 180.0 a | 137.5 a | 9.8 a | 8.3 a | 8.3 d |
| 4 | CXC Biocontrol | 1 | % v/v | A | Root Dip | 131.3 a | 199.0 a | 168.5 a | 9.5 a | 8.5 a | 17.5 bc |
| | CXC Biocontrol | 0.01 | % v/v | CEGIKM | Drench | | | | | | |
| | Kocide | 1.5 | lb/a | PRTVXZ | Foliar | | | | | | |
| 5 | CXC Biocontrol | 1 | % v/v | O-Z | Foliar | 123.8 a | 213.8 a | 153.8 a | 9.0 a | 9.0 a | 15.3 c |
| | Kocide | 1.5 | lb/a | O-Z | Foliar | | | | | | |
| 6 | CXC Biocontrol | 1 | % v/v | A | Root Dip | 99.5 a | 189.0 a | 157.5 a | 9.5 a | 7.8 a | 9.3 d |
| | CXC Biocontrol | 0.01 | % v/v | DHL | Drench | | | | | | |
| | Actigard | 0.75 | oz/a | QUY | Foliar | | | | | | |
| | Firewall | 200 | ppm pr | RVZ | Foliar | | | | | | |
| | Kocide | 1.5 | lb/a | PTX | Foliar | | | | | | |

No significant differences were reported in overall estimated marketable yield. Nevertheless, numerically the highest estimated yields were reported in plots treated with CXC Biocontrol solo or CXC Biocontrol with Kocide, either as a drench or a spray (Table 31).

TABLE 31
Estimated Marketable Weight (lbs/acre)

| Trt | Treatment | | Rate | Appl | Trt | EST MW/A | | | |
|---|---|---|---|---|---|---|---|---|---|
| No. | Name | Rate | Unit | Code | Descr. | Extra Large | Large | Medium | Total |
| 1 | Untreated Control | | | | | 10327.35 a | 34869.78 a | 11223.96 a | 56421.10 a |
| 2 | CXC Biocontol | 1 | % v/v | A | Root Dip | 12494.46 a | 37810.08 a | 12236.73 a | 62541.28 a |
| | CXC Biocontrol | 0.01 | % v/v | B-N | Drench | | | | |
| 3 | Kocide | 1.5 | lb/s | O-Z | Foliar | 10338.24 a | 32662.74 a | 10770.21 a | 53771.20 a |
| 4 | CXC Biocontrol | 1 | % v/v | A | Root Dip | 14175.15 a | 36060.42 a | 12694.11 a | 62929.69 a |
| | CXC Biocontrol | 0.01 | % v/v | CEGIKM | Drench | | | | |
| | Kocide | 1.5 | lb/a | PRTVXZ | Foliar | | | | |
| 5 | CXC Biocontrol | 1 | % v/v | O-Z | Foliar | 12893.76 a | 38880.93 a | 12273.03 a | 64047.73 a |
| | Kocide | 1.5 | lb/a | O-Z | Foliar | | | | |
| 6 | CXC Biocontrol | 1 | % v/v | A | Root Dip | 10360.02 a | 32125.50 a | 12149.61 a | 54635.14 a |
| | CXC Biocontrol | 0.01 | % v/v | DHL | Drench | | | | |
| | Actigard | 0.75 | oz/a | QUY | Foliar | | | | |
| | Firewall | 200 | ppm pr | RVZ | Foliar | | | | |
| | Kocide | 1.5 | lb/a | PTX | Foliar | | | | |

### Discussion

Overall, these results clearly demonstrate that compositions in accordance with the present invention are efficacious when applied as a drench, for controlling, suppressing and/or preventing infections from Clavibacter michiganensis subsp. michiganensis (Cmm) in tomato. The study further demonstrate that compositions in accordance with the present invention are compatible with existing commercial fungicides and that can be tank mixed together without a concern that the fungicide will denature, affect or damage our the composition of the invention. The compositions in accordance with the present invention have proven to be helpful when used alone and also in combination with existing means of canker control such as Kocide™.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a tomato” includes one or more tomato plants or tomato fruit (i.e., tomatoes) and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses, and such.

It is understood that the examples and embodiments described herein are for illustrative purposes only and that various modifications or changes in light thereof will be suggested to persons skilled in the art and are to be included within the present invention and scope of the appended claims.

1. A bacterial composition comprising an aqueous mixture of living Bacillus subtilis and living Bacillus pumilus, wherein said bacterial composition is formulated for soil drenching, and wherein said composition is effective for controlling, suppressing and/or preventing an infection from Clavibacter michiganensis subsp. michiganensis (Cmm) in tomato when applied directly to the base of a tomato plant.

2. The bacterial composition as defined in claim 1, wherein the Bacillus pumilus consists of strain ATCC® deposit number PTA-125304 and wherein the Bacillus subtilis consists of strain ATCC® deposit number PTA-125303.

3. The bacterial composition as defined in claim 1, further comprising at least one copper fungicide selected from the group consisting of copper sulfate, copper sulfate pentahydrate, copper hydroxide, copper oxychloride sulfate, cuprous oxide, copper octanoate, and mixtures thereof.

4. The bacterial composition as defined in claim 1, wherein application of said composition to at least one of a tomato plant, a tomato root, a tomato leaf, a tomato seed, a tomato stem and a tomato fruit provides at least one of the following benefits:
(i) increasing tomato yield;
(ii) increasing tomato size;
(iii) increasing gross return of tomatoes per acre; and
(iv) increasing yield for extra-large tomatoes.

5. A bacterial composition for controlling, suppressing and/or preventing an infection from Clavibacter michiganensis subsp. michiganensis (Cmm) in tomato, wherein said composition comprises an aqueous mixture of living Bacillus subtilis and living Bacillus pumilus, wherein each of said Bacillus subtilis and Bacillus pumilus are present in the composition at a concentration of about 107 to about 109 bacteria/ml, wherein the Bacillus pumilus consists of strain ATCC® deposit number PTA-125304 and wherein the Bacillus subtilis consists of strain ATCC® deposit number PTA-125303.

6. (canceled)

7. The bacterial composition as defined in claim 5, wherein said bacterial composition is formulated for soil drenching.

8. The bacterial composition as defined in claim 5, wherein said bacterial composition is formulated for foliar spray.

9. The bacterial composition as defined in claim 5, further comprising at least one copper fungicide selected from the group consisting of copper sulfate, copper sulfate pentahydrate, copper hydroxide, copper oxychloride sulfate, cuprous oxide, copper octanoate, and mixtures thereof.

10. The bacterial composition as defined in claim 5, wherein application of said composition to at least one of a tomato plant, a tomato root, a tomato leaf, a tomato seed, a tomato stem and a tomato fruit provides at least one of the following benefits:
(i) increasing tomato yield;
(ii) increasing tomato size;
(iii) increasing gross return of tomatoes per acre; and
(iv) increasing yield for extra-large tomatoes.

11. A method for controlling, suppressing and/or preventing an infection from Clavibacter michiganensis subsp. michiganensis (Cmm) in tomato, comprising:
-providing a bacterial composition comprising at least one of a mixture of Bacillus subtilis and Bacillus pumilus, a culture medium previously inoculated with Bacillus subtilis and Bacillus pumilus, a cell-free extract of Bacillus subtilis and Bacillus pumilus, and/or metabolites produced by Bacillus subtilis and Bacillus pumilus;
applying an effective amount of said bacterial as a root dip prior to planting; and
subsequently applying an effective amount of said bacterial composition as a root drench.

12. The method of claim 11, further comprising applying at least one of an herbicide, an insecticide, a fungicide, a bactericide, and a nutrient subsequent to said root dip.

13. The method of claim 11, further comprising applying at least one copper fungicide as a foliar spray after said root dip, and wherein said at least one copper fungicide selected from the group consisting of copper sulfate, copper sulfate pentahydrate, copper hydroxide, copper oxychloride sulfate, cuprous oxide, copper octanoate, and mixtures thereof.

14. A method for controlling, suppressing and/or preventing an infection from Clavibacter michiganensis subsp. michiganensis (Cmm) in tomato, comprising:
providing a bacterial composition comprising at least one of a mixture of Bacillus subtilis and Bacillus pumilus, a culture medium previously inoculated with Bacillus subtilis and Bacillus pumilus, a cell-free extract of Bacillus subtilis and Bacillus pumilus, and/or metabolites produced by Bacillus subtilis and Bacillus pumilus;
providing at least one copper fungicide selected from the group consisting of copper sulfate, copper sulfate pentahydrate, copper hydroxide, copper oxychloride sulfate, cuprous oxide, copper octanoate, and mixtures thereof; and
applying an effective amount of said bacterial composition to the roots of a tomato plant by root dipping and/or soil drenching; and
applying an effective amount of said copper fungicide by foliar spray.

15. The method of claim 14, wherein the copper fungicide is selected from the group consisting of Kocide 3000™, Kocide 2000TM, Kocide LF™, Kocide DF™, Kocide 101TM, Cuprofix™ Ultra 40 Disperss™, Nordox™, Nordox 75 WG™, Champ WG™, Cueva™, Badge SC™, Basic Copper 53TM, Bonide™, Camelot O™, Nu-Cop 3LTM, C-O-C-S WDG™, Previsto™, Badge X2TM, Badge MAXX™, Cuproxat™, Nordox 30 WG™, Copper-Count-N™ fungicide, Cuprablau Z 35 WP™ and Captain Jack′s™ liquid copper fungicide, Bordeaux™ mixture, Cuprofix Disperss™, Champion™, Nu-Cop™, Copper Count-NTM, Mastercop Instill™, Phyton™, and CS 2005TM.

16. The method of claim 15, wherein said method further comprises applying at least one of an herbicide, an insecticide, a second fungicide, a bactericide, and a nutrient.

17. The method of claim 14, wherein said bacterial composition comprises a mixture of living Bacillus subtilis and living Bacillus pumilus; wherein the Bacillus pumilus consists of strain ATCC® deposit number No. PTA-125304, and wherein the Bacillus subtilis consists of strain ATCC® deposit number PTA-125303 or ATCC® deposit number PTA-125302.

18-20. (canceled)

21. The method of claim 14, wherein the bacterial composition comprises Bacillus pumilus at a concentration of about 10⁹ CFU/ml to about 10⁶ CFU/ml and Bacillus subtilis at a concentration of about 10⁹ CFU/ml to about 10⁶ CFU/ml.

22. The method of claim 14, wherein the effective amount comprises applying between 10⁷ and 10⁹ CFU/cm³ of each of Bacillus pumilus and Bacillus subtilis.

23. The method of claim 14, wherein said method provides for at least one of the following benefits:
(i) increasing tomato yield;
(ii) increasing tomato size;
(iii) increasing gross return of tomatoes per acre; and
(iv) increasing yield for extra-large tomatoes;
wherein said benefit(s) is determined by comparing tomato contacted or not with said bacterial composition, and/or when comparing tomato contacted with said bacterial composition versus tomato contacted with a reference treatment.

24-28. (canceled)

29. A method for controlling, suppressing and/or preventing an infection from Clavibacter michiganensis subsp. michiganensis (Cmm) in tomato, comprising a combined application to a tomato plant of: (i) a bacterial composition comprising a mixture of living Bacillus subtilis and living Bacillus pumilus; and (ii) at least one of an herbicide, an insecticide, a fungicide, a bactericide, and a nutrient;
wherein said combined application provides for a synergistic efficacy in controlling. suppressing and/or preventing an infection from Clavibacter michiganensis subsp. michiganensis (Cmm) in tomato growing in the field.

30. The method of claim 29, wherein (ii) comprises at least one copper fungicide selected from the group consisting of copper sulfate, copper sulfate pentahydrate, copper hydroxide, copper oxychloride sulfate, cuprous oxide, copper octanoate, and mixtures thereof.

31. The method of claim 30, wherein the copper fungicide comprises Kocide™.

32-33. (canceled)
